(19) United States
(12) Patent Application Publication
(10) Pub. No.: US 2022/0211074 A1
(43) Pub. Date: Jul. 7, 2022

(54) STABILIZATION OF TUBER PROTEIN

(71) Applicant: COÖPERATIE KONINKLIJKE AVEBE U.A., Veendam (NL)

(72) Inventors: Robin Eric Jacobus SPELBRINK, Scharmer (NL); Maarten Hotse WILBRINK, Groningen (NL)

(21) Appl. No.: 17/613,366

(22) PCT Filed: May 25, 2020
(86) PCT No.: PCT/NL2020/050334

§ 371 Date: Nov. 22, 2021

(30) Foreign Application Priority Data
May 24, 2019 (EP) 19176594.0

(51) Int. Cl. A23J 3/14; A23J 1/00; A23K 10/35

(52) U.S. Cl. CPC A23J 3/14; A23K 10/35; A23J 1/006

(57) ABSTRACT

The invention provides a method for isolating root or tuber native protein, which method comprises at least the process steps of

a) processing a root or tuber to obtain root or tuber processing water comprising root or tuber native protein;
b) isolating said root or tuber native protein from said root or tuber processing water to obtain an aqueous solution comprising at least 5 wt. % of root or tuber native protein,
characterized in that all process steps are performed at a temperature lower than 40° C. This method results in root or tuber native protein with decreased tendency to gel, which has advantages for the processing of liquid protein solutions.

### BACKGROUND

There is increased demand for vegetarian and vegan analogues of conventional food products, due among others to the increased awareness of the environmental burden which comes with meat-derived food products. However, plant-based protein still cannot compete on various aspects with animal-derived products. One reason is that plant-based protein must often be isolated and processed, prior to being prepared into a food product. Such isolation may be in coagulated form, which is the most cost-effective method, but this also results in losing all functional properties of the protein. Native protein has various properties which make it more interesting for use in the food or feed industry than coagulated protein. Isolation of native protein is, however, more cumbersome.

During isolation of native protein, and processing of such protein into food products, it is preferred to use the protein in solution. Handling of powders is intrinsically more difficult than the handling of solutions, in particular on a large scale. Use of a protein solution has the benefit of easy dosing, efficient pumping, and faster cleaning, over use of protein powders. But in order to avoid handling excessive quantities of fluid, it is preferred to use concentrated, rather than diluted protein solutions.

One problem in the handling of root or tuber native protein solutions, in particular concentrated solutions such as solutions having a concentration of at least 5 wt. % protein, is that in such a solution, the protein has a tendency to form a gel. Once gelled, a concentrated solution is even more difficult to handle than a powder. A gelled protein solution can only be removed from a container with difficulty, and it can no longer be pumped or poured and is thus difficult to process. It is therefore desirable to identify ways to avoid gelling of concentrated protein solutions.

Conventionally, root or tuber native protein is isolated from root or tuber by various processes. Such processes normally comprise at least one step which requires an increased temperature, such as a temperature above 40° C.

For example, in tuber protein isolation from potato starch waste streams, separation of starch and fibers from the potato juice is generally followed by a step of concentrating the destarched juice in order facilitate and improve the efficiency of protein isolation. Cost-effective methods to achieve concentration generally comprise a heat treatment to at least 40° C. Furthermore, glycoalkaloid removal from liquids that contain native potato protein is generally performed at a temperature of 42° C.

In addition, potato starch processing streams generally are exposed to waste heat from mechanical devices such as graters, pumps, hydrocyclones, separators and decanters. This increases the temperature of the juice significantly. For example, sieving of the tuber pulp to separate the fibers from the juice can raise the temperature by 5-10° C. Hydrocyclones that separate the starch from the juice can yield a similar increase, and ultrafiltration of potato juice typically raises the temperature by 3° C. or more.

The present invention discloses that exposure of native protein to higher temperatures has the result of an increased tendency of the protein to gel, in particular when the protein is kept in solution for prolonged time and/or at high concentration. Consequently, the invention provides a method to isolate native root or tuber protein, with which method protein gel formation can be avoided.

### DESCRIPTION OF FIGURES

FIG. 1: dynamic viscosity of a 16 wt. % protease inhibitor protein solution upon long-term storage, after heating to various temperatures

FIG. 2 viscosity development upon long-term storage under ambient conditions as a function of pH and concentration. A: 16 wt. %, B: 20 wt. %, C: 24 wt. %.

FIG. 3: Dynamic viscosity as a function of total temperature exposure

### DETAILED DESCRIPTION

The invention discloses a method for isolating root or tuber native protein, which method comprises at least the process steps of

a) processing a root or tuber to obtain root or tuber processing water comprising root or tuber native protein;
b) isolating said root or tuber native protein from said root or tuber processing water to obtain an aqueous solution comprising at least 5 wt. % of root or tuber native protein,
characterized in that all process steps are performed so as to prevent exposure of native protein to a temperature higher than 40° C.

The presently claimed invention pertains to a method comprising at least the process steps of

a) processing a potato to obtain potato processing water comprising native potato protease inhibitor;
b) isolating a native potato protease inhibitor from said potato processing water to obtain an aqueous solution of pH 2.5-4.0 comprising at least 5 wt. % native potato protease inhibitor as the sole type of potato protein, characterized in that all process steps are performed so as to prevent exposure of native protease inhibitor to a temperature higher than 40° C.

The present invention is directed to a concentrated aqueous solution of root or tuber native protein, as well to protein obtained from such a solution. Concentrated, in this regard, means that the aqueous solution comprises at least 5 wt. %, preferably at least 8 wt. %, more preferably at least 12 wt. %, even more preferably at least 15 wt. %. Preferably however, protein concentration is lower than 24 wt. %, preferably lower than 23 wt. %, more preferably lower than 22 wt. %. Preferred concentrated protein solutions have a concentration of 5-24 wt. %, preferably 8-20 wt. %.

The aqueous solution which is obtained by the present method has the advantage of having higher stability than other concentrated root or tuber native protein solutions. Stability, in this regard, is physical stability, which in the present context essentially means that the solution retains its physical characteristics, most notably viscosity, color and the like. Viscosity in the present context is dynamic viscosity, determined using a Thermo HAAKE MARS model III rheometer at 25° C., using the protocol set forth in the Examples.

It has been found that by avoiding high temperature exposure during the isolation of root or tuber native protein, a concentrated solution of the thus obtained protein exhibits less gelling than a solution of the same protein at the same concentration, which has been subjected to high temperatures. Thus, the concentrated native protein solution remains fluid and workable for prolonged periods of time. This has advantages for the processing of liquid protein solutions. Fluid and/or workable, in this context, means that the dynamic viscosity of the concentrated protein solution is at most 10 Pa·s, preferably at most 5 Pa·s. A concentrated native protein solution obtained according to the present method can be kept for weeks or months under ambient conditions while retaining its physical characteristics, and remaining fluid and workable.

Without wishing to be bound by theory, it is presently believed that higher temperatures than 40° C. lead to breaking of disulfide bonds which stabilize the protein three-dimensional structure. Protein which is structurally intact is less susceptible to gelling. Protein in which a significant fraction of disulfide bonds is broken, is more susceptible to gelling. Therefore, avoiding high temperatures during isolation of native protein has the effect that a thus obtained protein is less susceptible to gelling.

Higher protein concentration also leads to an increased tendency for gelling. Therefore, it is particularly important that protein present in a concentrated solution has not been exposed to high temperatures as herein defined.

Concentrated solutions, which normally would be susceptible to gelling, are capable of retaining low viscosity by applying an isolation process in which the all process steps are performed so as to prevent exposure of native protein to a temperature higher than 40° C. The invention thus also discloses a method for obtaining a stable concentrated protein solution, or a method for stabilizing a protein solution, which method comprises the steps set forth herein, characterized in that all process steps are performed so as to prevent exposure of native protein to a temperature higher than 40° C.

It is unexpected that applying an isolation process in which protein has not been exposed to temperatures higher than 40° C. avoids gel formation in time. Most substances which have a tendency to gel do so when cooling down after being exposed to high temperatures; for most substances, increasing the temperature avoids gel formation, or even melts the gel. In the present case of concentrated solutions of root or tuber native protein, lower temperature prevents gel formation during storage of the protein solution. Thus, processing of a native protein solution should be performed at temperatures of lower than 40° C., and storage of concentrated protein solutions should preferably be at low temperature, such as at most 25° C., preferably at most 20° C., more preferably at most 15° C., and even more preferably at most 5° C., in order to avoid gel formation.

In addition, it has been found that the presence of reducing agents also leads to breaking of disulfide bonds, thereby enhancing the susceptibility of the protein towards gelling. The addition of reducing agents such as SO₂ or bisulfite is however common practice in the processing of potatoes to prevent browning of potato juice.

In some embodiments therefore, the present invention provides methods for isolating protein, for obtaining a stable concentrated protein solution, or for stabilizing a protein solution, wherein the quantity of sulfite in the final solution is below 800 ppm, preferably below 500 ppm, more preferably below 200 ppm, even more preferably below 100 ppm. Solutions with this concentration of sulfite also display enhanced viscosity stability. Preferably, the enhanced viscosity stability is achieved by also preventing exposure of protein to a temperature higher than 40° C., but the insight that enhanced viscosity stability is obtained by applying low concentrations of sulfite as described is independent from the insight that enhanced viscosity stability can be attained by avoiding exposure of protein to temperatures higher than 40° C.

The invention also pertains to isolated native protein which is structurally intact. e.g. in terms of amino acid order, three dimensional structure and functional properties such as enzymatic activity, solubility in water, and/or texturizing properties, either as a concentrated solution or as a protein powder after drying said concentrated solution. The protein is characterized by a higher quantity of disulfide bonds in the protein, than conventionally processed protein.

Root or tuber native protein, in the present context, is native protein isolated from a root or a tuber. It may be referred to as root- or tuber derived native protein, or as native protein from root or tuber. The term root or tuber is to be given its regular meaning, and refers to any root- or tuber as found on any type of root or tuber plant. Preferably, a root- or tuber in the present context is an edible root or tuber, which may be grown in the context of human food production. Although normally, root or tuber native protein refers to a single type of protein from one type of root or tuber, or to a particular protein fraction from one type of root or tuber, in special cases, root or tuber native protein may comprise a mixture of native protein derived from two or more types of root or tuber.

Preferably, root or tuber in this context comprises potato (Solanum tuberosum), sweet potato (Ipomoea batatas), cassava (including Manihot esculenta, syn. M. utilissima, also called manioc, mandioca or yuca, and also including M. palmata, syn. M. dulcis, also called yuca dulce), yam (Dioscorea spp), and/or taro (Colocasia esculenta). More preferably, the root or tuber is a potato, sweet potato, cassava or yam, even more preferably the root or tuber is a potato, sweet potato or cassava, even more preferably the root or tuber is a potato or sweet potato, and most preferably the root or tuber is a potato (Solanum tuberosum).

Thus, preferred root or tuber native protein is native potato protein, native sweet potato protein, native cassava protein, native yam protein, and/or native taro protein.

Preferably the root or tuber native protein comprises native root or tuber protease inhibitor, a native root or tuber patatin or a mixture comprising protease inhibitor and patatin. A mixture comprising protease inhibitor and patatin can be called a total root or tuber protein.

Most preferably, the root or tuber native protein is derived from potato (Solanum tuberosum), i.e. comprises native potato protein protease inhibitor, native potato patatin, or a mixture comprising potato-derived protease inhibitor and potato-derived patatin. Such a mixture can be referred to as total potato protein.

Native, in the present context, is a term conventionally known term in the art of protein processing. Protein as it occurs in its natural environment is considered native. Upon isolation of protein from its natural environment protein readily degrades and/or denatures, that is, loses its three-dimensional structure and functionality at least to some degree. Native protein is thus understood to mean protein which is not significantly degraded and which is not significantly denatured. Native protein in the present context is thus protein which essentially retains its natural enzymatic activity and its natural three-dimensional structure.

The degree of native-ness of protein can be tested by a solubilization experiment. Non-native protein is considerably less soluble in water than native protein. Protein solubility can be determined by dispersing protein in water, dividing the resulting liquid into two fractions and exposing one fraction to centrifugation at 800 g for 5 minutes to create a pellet of non-dissolved material and recovering the supernatant. By measuring the protein content in the supernatant and in the untreated solution, and expressing the protein content of the supernatant as a percentage of that in the untreated solution, the solubility is determined. Convenient methods to determine the protein content are via the Sprint Rapid Protein Analyser (CEM), by measuring the absorbance at 280 nm. In the present context, protein is considered native if the solubility of the protein is at least 80%, preferably at least 85%, more preferably at least 90%, even more preferably at least 95, most preferably at least 98%.

Patatin, as defined herein, is a root or tuber protein, preferably a potato protein, which is an acidic glycoprotein which in the tuber functions as storage protein. In the root and tuber processing industry, it is generally known which of the root or tuber proteins is considered patatin. Patatin, in the present context, refers to a root or tuber protein fraction in which at least 80 wt. %, preferably at least 85 wt. %, more preferably at least 90 wt. % of all protein has a molecular weight of more than 35 kDa as determined by SDS-page.

Protease inhibitor, as defined herein, is a root or tuber protein, preferably a potato protein, which in native form is capable of inhibiting the protease activity of proteases. It is common general knowledge which root or tuber protein is considered a protease inhibitor. Protease inhibitor, in the present context, refers to a root or tuber protein fraction in which at least 80 wt. %, preferably at least 85 wt. %, more preferably at least 90 wt. % of all protein has a molecular weight of at most 35 kDa as determined by SDS-page.

SDS-page (sodium dodecyl sulfate polyacrylamide gel electrophoresis) is a generally known technique for determining the molecular weight of a protein.

Isolating root or tuber native protein, in the present context, means that protein which is naturally present in native form in root or tuber as defined above, is extracted from said root or tuber without significantly affecting the protein. Thus, native protein is not significantly degraded and is not significantly denatured. That is, the amino acid order, the three dimensional structure and the functional properties are essentially intact, in comparison to the protein as it occurs in the root or tuber.

In preferred embodiments, the process of the invention is applied on a root or tuber native protein as the sole type of root or tuber protein. A solution comprising root or tuber patatin as the sole type of root or tuber protein comprises no other types of root or tuber protein other than defined under patatin. Likewise, a solution comprising root or tuber protease inhibitor as the sole type of root or tuber protein comprises no other types of root or tuber protein other than defined under protease inhibitor. In these embodiments, the invention allows to obtain a pure root or tuber patatin isolate, or a pure root or tuber protease inhibitor isolate, such as for example a potato protease inhibitor isolate. However, the process may also be performed in order to obtain a root or tuber native protein isolate comprising protease inhibitor and patatin.

A solution comprising root or tuber patatin as the sole type of root or tuber protein may be obtained by known methods, such as by selective absorption of the protease inhibitors. A solution comprising root or tuber protease inhibitor as the sole type of root or tuber protein may be obtained by known methods, such as by selective precipitation of patatin and/or selective absorption of patatin. How to achieve selective absorption of patatin or protease inhibitor has been described in WO2008/069660.

A concentrated protein solution may further comprise various other root- or tuber derived constituents, such as starch, fiber, cell debris, sugars, salts and the like, as is well-known in the art. Preferably however, the concentrated protein solution comprises a more or less pure native protein, for example comprising at least 50 wt. % protein, based on total dry matter (DM), preferably at least 75 wt. % DM, more preferably at least 85 wt. % DM, even more preferably at least 90 wt. % DM, and most preferably at least 95 wt. % DM protein. Protein quantities can be determined using a CEM Sprint Rapid protein analyzer.

In order to obtain a concentrated root or tuber native protein solution, at least two steps must be performed. Root or tuber must be processed so as to obtain root or tuber processing water comprising native protein (step a), and said native protein must be isolated from said processing water to obtain a concentrated solution of root or tuber native protein (step b). Both steps are essentially known steps. It has been found however that these steps and any other optional steps which may be applied should be performed so as to prevent exposure of the native protein to a temperature higher than 40° C., in order to obtain a concentrated protein solution with stable physical characteristics.

In the present context, any protein solution or suspension should thus not be processed at a temperature higher than 40° C., and processing of solution to powder should not be performed at temperatures higher than 40° C.

Use of higher processing temperatures is limited to situations wherein protein is not affected by the application of heat. For example, whole potatoes can be subjected to brief heat exposure, without affecting the protein inside the potato. This is because the inside of the potato does not heat fast enough to exceed 40° C. in the context of such treatments. Thus, brief heat exposure steps such as blanching, sterilization and steam peeling can be steps comprised in the present process, despite such steps being performed at a temperature higher than 40° C.

Preferably however, all process steps to isolate protein in the present context are performed at a temperature below 40° C., and/or none of the process steps apply a temperature higher than 40° C.

In order to attain the advantageous effects described herein, it is preferred that the total temperature exposure of the protein is kept to a minimum. The total temperature exposure is defined as the product of time (in hour) and temperature (in ° C., because water freezes at 0° C.), and is called the TTE. Preferably, the TTE is less than 250° C. hr, more preferably less than 200° C.·hr.

In step a, root or tuber is processed to obtain root or tuber processing water comprising native protein. Native protein is protein as it occurs in the root or tuber of origin, which is dissolved in the processing water. Such processing comprises for example pulping, mashing, rasping, grinding, pressing or cutting of the root or tuber, and optionally a combination with water.

In preferred embodiments, processed (pulped, mashed, rasped, ground, pressed, cut and the like) root or tuber is combined with water to obtain root or tuber juice which comprises native protein as well as starch. Such processing water may be subjected to a step of starch removal, for example by decanting, cycloning, or filtering as is known in the art, to obtain a root or tuber processing water comprising native protein. In such embodiments, root or tuber processing water is preferably a waste product from the starch industry, such as potato fruit juice (PFJ) as obtained after starch isolation.

In other preferred embodiments, root or tuber is processed by cutting to form shapes of root or tuber which are the basis for processed root- or tuber products like for example chips and fries. Such cutting, when performed in the presence of water, results in root or tuber processing water comprising root or tuber native protein.

In one embodiment, root or tuber may be processed by a water jet stream to cut the root or tuber. In another embodiment, root or tuber may be processed by cutting knives, for example in the presence of water. The water which results from such cutting processes comprises root or tuber native protein, and consequently is a root or tuber processing water in the meaning of step a.

Processing according to step a can be done on whole root or tuber, or on root or tuber which has been peeled.

In step b, root or tuber native protein is isolated from the root or tuber processing water to obtain a concentrated aqueous solution comprising root or tuber native protein. Methods to achieve this are essentially known. Preferred methods to isolate protein from the processing water include ultrafiltration, diafiltration, isoelectric precipitation, heat precipitation, complexation, adsorption or chromatography, or a combination of two or more of these methods. Optionally, said isolation step is followed by a concentration step, such as by ultrafiltration, diafiltration, reversed osmosis, or freeze concentration, to obtain said concentrated solution of root or tuber native protein.

A preferred technique for isolating native protein is the use of ultrafiltration (UF). Ultrafiltration separates solutes in the molecular weight range of 5 kDa to 500 kDa and can therefore be used for the separation of protein from low molecular weight solutes. UF membranes may have pores ranging from 1 to 20 nm in diameter. Preferred UF membranes are anisotropic UF-membranes. Preferably, the ultrafiltration membrane comprises polyvinylidenefluoride, regenerated cellulose, a polyethersulphone (PES) or a polysulphone (PS). An UF membrane can be implemented as tubular, spiral wound, hollow fibre, plate and frame, or as cross-rotational induced shear alter units.

The ability of an ultrafiltration membrane to retain macromolecules is traditionally specified in terms of its molecular cut-off (MWCO). A MWCO value of 10 kDa means that the membrane can retain from a feed solution 90% of the molecules having molecular weight of 10 kDa. Preferred MWCO's in the present context are 1-300 kDa membranes, preferably 2-250 kDa, more preferably 3-200 kDa, more preferably 5-150 kDa. In some embodiments, a 5-20 kDa membrane is used, preferably 5-10 kDa. In other embodiments, a 30-200 kDa membrane is used, preferably 50-150, more preferably 80-120.

Protease inhibitor is preferably obtained using a PES or PS membrane with a molecular weight cut-off as defined above. Protease inhibitor can be subjected to UF at a pH of 3-7, preferably 3.2-4.5.

Patatin is preferably obtained using a PES, a PS or a regenerated cellulose membrane with a molecular weight cut-off as defined above. Patatin can be subjected to UF at a pH value of 4.0-8.0, preferably pH 6.0-7.5. After removal of impurities the pH may be increased to pH 7-10 to enable high fluxes through the membranes. In the context of the present invention, the isolation step is preferably followed by a pH adjustment step, as described below.

A further preferred technique for protein isolation is diafiltration (DF). Diafiltration may be achieved through the same membranes as ultrafiltration, against water or a salt solution, preferably a salt solution. Diafiltration is preferably followed by a concentration step, such as ultrafiltration or reverse osmosis, preferably ultrafiltration.

In a preferred embodiment, the root or tuber native protein as obtained after concentration has a protein content of more than 75% of the dry matter content. The protein content herein is defined as Kjeldahl nitrogen content times 6.25. Preferably the protein content is more than 80%, more preferably more than 85%, even more preferably more than 90%, and even more preferably more than 95%, expressed on dry matter.

Isolation of protein may be performed for example as described in EP 2 083 634, WO2014/011042 by adsorption and elution, or by other methods known in the art.

Root or tuber native protein may furthermore be isolated by chromatography, such as cation exchange chromatography, anion exchange chromatography, as is known in the art. Other techniques to isolate root or tuber native protein include isoelectric focusing, isoelectric precipitation and complexation, as is known in the art.

It is preferred that the present method is applied on an industrial scale. Thus, the present method is preferably operated to result in at least 5 kg of protein per hour, more preferably at least 25 kg protein per hour, even more preferably at least 50 kg protein per hour.

The present method may comprise further process steps, as long as such process steps are executed so as to prevent exposure of protein to a temperature higher than 40° C., as defined herein.

In one embodiment, the method further comprises a step of glycoalkaloid removal. This may be achieved by known means, such as adsorption to activated carbon, hydrophobic resins or various types of clay, by chromatography, by acid extraction, by enzymatic conversion or by fermentation. Exemplary techniques are described in WO 2008/056977 and WO 2008/069651.

In another embodiment, the method may comprise a step of flocculation, in order to remove among others lipids and microparticles. Flocculation may for example be performed as described in WO 2016/036243.

The method may furthermore comprise a step of filtration, preferably microfiltration.

Furthermore, the method may comprise various other known steps to aid in the isolation of protein, starch, or of other root- or tuber constituents. For example, the method may comprise one or more filtration steps, one or more pH adjustment steps, one or more centrifugation steps, as well as cycloning. Adjustment of the pH of a solution can be performed by the addition of acid or base, as is well known in the art. Suitable acids are for example hydrochloric acid, citric acid, acetic acid, formic acid, phosphoric acid, sulfuric acid and lactic acid, and suitable bases are for example sodium or potassium hydroxide, ammonium chloride, sodium or potassium carbonate, oxides and hydroxides of calcium and magnesium.

In order to stabilize the physical characteristics of the concentrated root or tuber native protein solution, it is further preferred that the concentrated root or tuber native protein solution has a pH higher than 2.5, preferably higher than 2.75. This embodiment is suitable for any root or tuber native protein, but is in particular suitable for root or tuber protease inhibitor, in particular potato protein protease inhibitor. This pH value can be attained by appropriate addition of acid or base, as described.

In order to stabilize the physical characteristics of the concentrated root or tuber native protein solution it is further preferred that the concentrated root or tuber native protein solution has a pH lower than 4.0, preferably lower than, 3.5, preferably lower than 3.25, more preferably lower than 3.0. This embodiment is suitable for any root or tuber native protein, but is in particular suitable for root or tuber derived patatin and protease inhibitor, in particular potato-derived protease inhibitor. This pH value can be attained by appropriate addition of acid or base, as described.

For stabilizing the physical characteristics of a concentrated root or tuber native protein solution comprising protease inhibitor and/or patatin, it is much preferred that the pH of the concentrated solution is higher than 2.5, preferably higher than 2.75, and lower than 3.5, preferably lower than 3.25, more preferably lower than 3.0. This pH value can be attained by appropriate addition of acid or base, as described.

Stabilization of the protein solution at a pH of less than 4.0, preferably 2.5-4.0, more preferably 2.5-3.5, has the additional benefit of reducing microbial growth, and thus increasing microbial stability of the solution.

It is furthermore preferred that a concentrated root or tuber native protein solution comprising protease inhibitor and/or patatin, has a sulfite concentration of less than 800 ppm, preferably less than 500 ppm, preferably less than 200 ppm, preferably less than 100 ppm.

In much preferred embodiments, the method further comprises a step of drying the concentrated solution. Drying steps are well known, and may comprise evaporation, spray drying, freeze drying, and the like. Such drying results in a root or tuber native protein powder, which is less susceptible to gelling.

The invention is furthermore directed to native protease inhibitor, wherein the quantity of disulfide bridges is 4-8 g/kg, expressed as g cysteine present as disulfide bridge per kg protein.

The invention is furthermore directed to native patatin, wherein the quantity of disulfide bridges is 6-24 g/kg, expressed as g cysteine present as disulfide bridge per kg protein.

The invention is furthermore directed to root or tuber native protein, wherein the quantity of disulfide bridges is 4-24 g/kg, preferably 6-24 g/kg, more preferably 12-24 g/kg, even more preferably 18 g/kg, expressed as g cysteine present as disulfide bridge per kg protein. The amount of disulfide bridges depends on the class of native protein. Protease inhibitor comprises 4-8 g/kg disulfide bridges, whereas patatin comprises 6-24 g/kg disulfide bridges. The quantity of disulfide bridges is preferably 40-235 mmol/kg protein, more preferably 60-235 mmol/kg protein, even more preferably 120 mmol/kg protein, even more preferably 180 mmol/kg protein. Protein with the recited quantity of disulfide bridges is less susceptible to gelling, in particular when present in a concentrated solution.

The quantity of disulfide bridge per unit protein can be measured by determination of the free thiol concentration, followed by reduction of the disulfide bonds. Subsequent quantification of the additionally exposed thiols provides the quantity of disulfide bridges which were present in the sample. This can be expressed as gram cysteine per kg protein, or mol cysteine per kg protein, as required.

Free thiols can be quantified spectrophotometrically, by reacting the sample with for example Ellmans reagens (5,5′-dithiobis (2-nitrobenzoic acid), DTNB), 4,4′-dithiodipyridine (4-DPS), Monobromobimane (mBBr) or a benzofurazan such as 7-fluorobenzo-2-oxa-1,3-diazole-4-sulfonate (SBD-F) or 4-(aminosulfonyl)-7-fluoro-2,1,3-benzoxadiazole (ABD-F).

Preferably, said protein has a glycoalkaloid content of at most 200 mg/kg DM, more preferably at most 100 mg/kg DM, even more preferably 50 mg/kg DM. Further preferably, said protein has a protein content, as determined by Sprint, of at least 50 wt. % protein, based on total dry matter (DM), preferably at least 75 wt. % DM, more preferably at least 85 wt. % DM, even more preferably at least 90 wt. % DM, and most preferably at least 95 wt. % DM protein.

In much preferred embodiments, said native protein is a native potato protein protease inhibitor, a native potato-derived patatin, or a native total potato protein comprising protease inhibitor and patatin.

The invention furthermore provides a food or feed product, comprising root or tuber native protein as defined above. Preferably, the food or feed product comprises 0.1-10 wt. % of native protein, more preferably 0.5-5 wt. %. Such food or feed products have the advantage that root or tuber native protein can be included for conventional reasons, whereas providing less tendency to gel, in particular when present at high concentration.

The invention as regards root or tuber patatin can furthermore be described in the following terms:

1. A method for isolating root or tuber native patatin, which method comprises at least the process steps of
a) processing a root or tuber to obtain root or tuber processing water comprising root or tuber native patatin;
b) isolating said root or tuber native patatin from said root or tuber processing water to obtain an aqueous solution of pH less than 3.5 comprising at least 5 wt. % of root or tuber native patatin as the sole type of potato protein,
characterized in that all process steps are performed so as to prevent exposure of native protein to a temperature higher than 40° C.
2. A method according to term 1, wherein the method is operated to result in at least at least 5 kg of protein per hour.
3. A method according to term 1 or 2, wherein the pH of the aqueous solution is higher than 2.5, preferably higher than 2.75.
4. A method according to any of terms 1-3, wherein the pH of the aqueous solution is lower than 3.25.
5. A method according to any of terms 1-4, wherein said processing to obtain root or tuber processing water comprises pulping, mashing, rasping, grinding, pressing or cutting of the root or tuber, and optionally a combination with water.
6. A method according to any of terms 1-5, wherein said processing further comprises a step of starch removal.
7. A method according to any of terms 1-6, wherein said isolating of root or tuber native patatin comprises a step of ultrafiltration, diafiltration, adsorption, precipitation or chromatography.
8. A method according to any of terms 1-7, wherein the method further comprises a glycoalkaloid removal step.
9. A method according to any of terms 1-8, wherein the root or tuber native patatin is native potato patatin, native sweet potato patatin, native cassava patatin, native yam patatin, or native taro patatin.
10. A method according to any of terms 1-9, wherein the root or tuber native patatin comprises native potato patatin.
11. A method according to any of terms 1-10, wherein the aqueous solution is dried to obtain a root or tuber native patatin powder.
12. A method according to any of terms 1-11, wherein the total temperature exposure (TTE) is less than 250° C. hr, preferably less than 200° C.·hr.
13. A root or tuber native patatin obtainable by any of terms 1-12, wherein the quantity of disulfide bridges is 6-24 g/kg, expressed as g cysteine present as disulfide bridge per kg protein.
14. A root or tuber native patatin according to term 13, which has a glycoalkaloid content of at most 200 mg/kg protein.
15. A human food product or an animal feed product comprising a root or tuber native patatin according to any of terms 13-14.

The invention as regards root or tuber total protein can furthermore be described in the following terms:

1. A method for isolating root or tuber native protein, which method comprises at least the process steps of
a) processing a root or tuber to obtain root or tuber processing water comprising root or tuber native protein;
b) isolating said root or tuber native protein from said root or tuber processing water to obtain an aqueous solution of pH 2.5-4.0 comprising at least 5 wt. % of root or tuber native protein, wherein the root or tuber native protein comprises native patatin and native protease inhibitor, characterized in that all process steps are performed so as to prevent exposure of native protein to a temperature higher than 40° C.
2. A method according to term 1, wherein the method is operated to result in at least at least 5 kg of protein per hour.
3. A method according to term 1 or 2, wherein the pH of the aqueous solution is higher than 2.75.
4. A method according to any of terms 1-3, wherein the pH of the aqueous solution is lower than 3.5.
5. A method according to any of terms 1-4, wherein said processing to obtain root or tuber processing water comprises pulping, mashing, rasping, grinding, pressing or cutting of the root or tuber, and optionally a combination with water.
6. A method according to any of terms 1-5, wherein said processing further comprises a step of starch removal.
7. A method according to any of terms 1-6, wherein said isolating of root or tuber native protein comprises a step of ultrafiltration, diafiltration, adsorption, precipitation or chromatography.
8. A method according to any of terms 1-7, wherein the method further comprises a glycoalkaloid removal step.
9. A method according to any of terms 1-8, wherein the root or tuber native protein is native potato protein, native sweet potato protein, native cassava protein, native yam protein, or native taro protein.
10. A method according to any of terms 1-9, wherein the aqueous solution is dried to obtain a root or tuber native protein powder.
11. A method according to any of terms 1-10, wherein the total temperature exposure (TTE) is less than 250° C. hr, preferably less than 200° C.·hr.
12. A root or tuber native protein comprising native protease inhibitor and native patatin obtainable by any of terms 1-11, wherein the quantity of disulfide bridges is 4-24 g/kg, expressed as g cysteine present as disulfide bridge per kg protein.
13. A root or tuber native protein according to term 12, which has a glycoalkaloid content of at most 200 mg/kg protein.
15. A human food product or an animal feed product comprising a root or tuber native protein according to any of terms 12-13.

For the purpose of clarity and a concise description features are described herein as part of the same or separate embodiments, however, it will be appreciated that the scope of the invention may include embodiments having combinations of all or some of the features described. The invention will now be illustrated by the following, non-limiting examples.

### EXAMPLES

Dynamic viscosity is determined using a Thermo HAAKE MARS model III rheometer, equipped with Z40 rotor DIN 53019/ISO 3219 (low inertia for RV/RS/MARS) and Cup Z40 (Ø=40 mm), using HAAKE RheoWin software. Dynamic viscosity (η) of protein concentrates is measured at 25° C., by applying a protocol in three phases:

1) Oscillation (f=0.100 Hz)

2) Increasing shear rate (0.1-1.000 l/s)

3) Decreasing shear rate (1,000-0.1 l/s)

The value determined in step 2) at the lowest shear rate is taken as a measure of dynamic viscosity (YI) in Pa s. Solutions were considered workable and fluid when the viscosity was 10 Pa·s or less.

Protein concentrations were determined using a CEM Sprint Rapid protein analyzer that was calibrated against Kjeldahl measurements. Sprint measures the loss of signal of a protein-binding dye. The higher the loss, the more protein is present. This system is calibrated using Kjeldahl measurements on extensively dialysed protein samples so that all nitrogen that is detected will originate from protein and not from free amino acids, peptides or other nitrogen sources. The nitrogen-number is then converted into a protein content by multiplying with 6.25.

### Example 1

Potato was ground and subjected to starch removal with all process steps executed at temperatures between 15 and 25° C. The resulting potato fruit juice was processed using method 2 described in EP 1 920 662, resulting in an isolated protease inhibitor.

The isolated potato protein was obtained at a concentration of 16 wt. %, the pH was adjusted to 2.8, and the protein solution was exposed to temperatures of 35, 39, 43 and 47° C. for 4 hours and stored under ambient conditions (20-25° C.). The viscosity was determined prior to storage (week 0), and after 1, 2, 4, 9, 18 and 52 weeks.

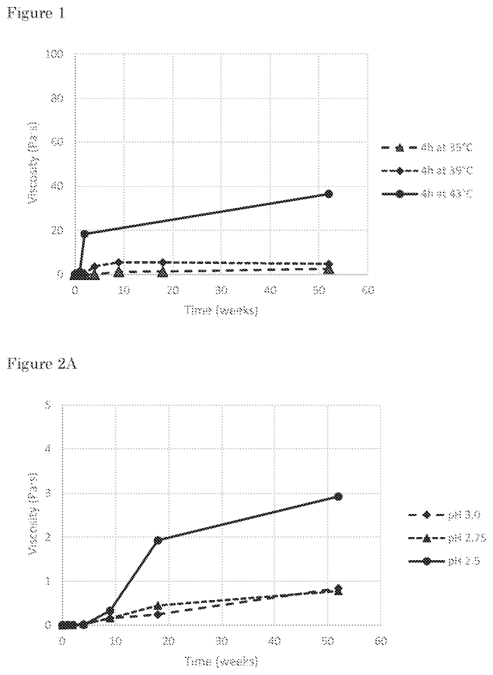

The results show that material which has not been subjected to temperatures above 40° C. retain their physical stability (in terms of dynamic viscosity) and remain fluid and workable. Material which is subjected to temperatures above 40° C. forms a gel, and thus cannot be stored or shipped, as it is no longer processable upon arrival. Also see FIG. 1.

TABLE 1
Dynamic viscosity ([Pa · s]) of a 16 wt. % potato protease
inhibitor concentrate with pH 2.8 after different heat treatments
under long term storage at ambient temperature.

| time ([weeks]) | 0 | 1 | 2 | 4 | 9 | 18 | 52 |
|---|---|---|---|---|---|---|---|
| 4 h at 35° C. | 0.00 | 0.01 | 0.02 | 0.07 | 1.3 | 1.3 | 2.6 |
| 4 h at 39° C. | 0.00 | 0.02 | 0.20 | 3.7 | 5.6 | 5.5 | 4.8 |
| 4 h at 43° C. | 0.01 | 1.3 | 18 | — | — | — | 36.6 |

| | |
|---|---|
| 4 h at 47° C. | solid gel formation upon heating, |
| | no viscosity determined |

### Example 2

The potato protease inhibitor obtained in Example 1 at 16 wt. % concentration was exposed to temperatures of 30, 33, 36 and 39° C. for periods of 3, 6 or 12 hours. Subsequently, the protein was stored under ambient conditions (20-25° C.), and the viscosity was determined one day later.

The results show that higher temperature and longer exposure time results in an increased tendency to gel. Not only the process temperature should be minimized, but also the time during which the protein is exposed to the higher temperatures should be minimized.

TABLE 2
Dynamic viscosities ([Pa · s]) of a 16 wt. % potato protease inhibitor
concentrate with pH 2.8 one day after different heat treatments

temp:

| time | 30° C. | 33° C. | 36° C. | 39° C. |
|---|---|---|---|---|
| 0 | 0.02 | 0.02 | 0.02 | 0.02 |
| 3 | 0.2 | 0.7 | 6.8 | 2.2 |
| 6 | 0.5 | 6.1 | 11.6 | 20.6 |
| 12 | 15.3 | 42.8 | 62.5 | 83.4 |

### Example 3

The potato protease inhibitor obtained in Example 1 was concentrated to obtain 16, 20 and 24 wt. % protein concentrates by ultrafiltration. Subsequently, the pH was adjusted to values of 2.5, 2.75 and 3.0, and the solutions were stored under ambient conditions (20-25° C.). The viscosity of the solution was determined immediately, and after 1, 2, 4, 9, 18 and 52 weeks.

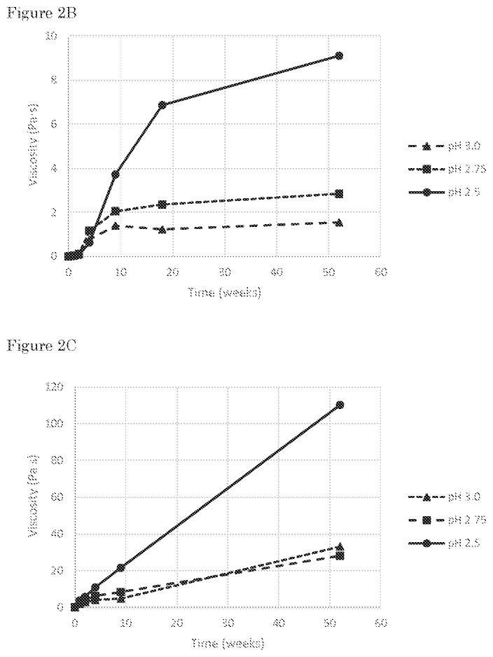

The results show that the tendency of this protein to gel is pH and concentration dependent. A pH higher than 2.5, preferably higher than 2.75 is preferred. Also, the tendency to gel is concentration dependent. Protein concentration is preferably lower than 24 wt. %, in order to remain fluid and processable. Also see FIG. 2a (16 wt. %), 2b (20 wt. % and 2c (24 wt. %).

TABLE 3
Dynamic viscosity ([Pa · s]) of a 16, 20 and 24 wt. % potato protease
inhibitor concentrate upon storage at different pH values

| | 0 | 1 | 2 | 4 | 9 | 18 | 52 |
|---|---|---|---|---|---|---|---|
| pH 2.5, 16 wt. % | 0.01 | 0.01 | 0.01 | 0.01 | 0.3 | 1.9 | 2.9 |
| pH 2.5, 20 wt. % | 0.01 | 0.02 | 0.08 | 0.6 | 3.7 | 6.9 | 9.1 |
| pH 2.5, 24 wt. % | 0.02 | 8.8 | 5.8 | 10.9 | 21.6 | — | 110 |
| pH 2.75, 16 wt. % | 0.00 | 0.01 | 0.01 | 0.03 | 0.2 | 0.5 | 0.8 |
| pH 2.75, 20 wt. % | 0.01 | 0.04 | 0.12 | 1.16 | 2.1 | 2.4 | 2.8 |
| pH 2.75, 24 wt. % | 0.03 | 2.8 | 4.2 | 6.9 | 8.4 | — | 28 |
| pH 3.0, 16 wt. % | 0.01 | 0.01 | 0.01 | 0.03 | 0.2 | 0.3 | 0.8 |
| pH 3.0, 20 wt. % | 0.01 | 0.03 | 0.09 | 0.76 | 1.4 | 1.2 | 1.5 |
| pH 3.0, 24 wt. % | 0.04 | 1.9 | 2.9 | 4.1 | 4.9 | — | 33 |

### Example 4

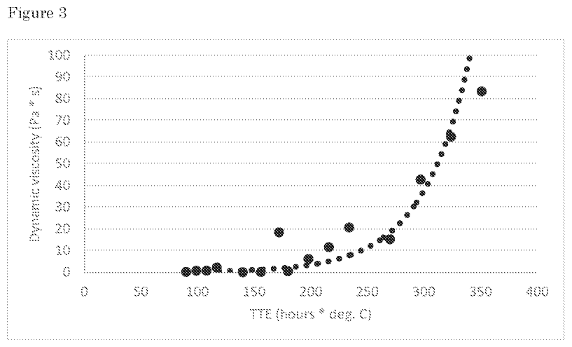

The results of Examples 1, 2 and 3 were pooled to gain insight in the relevance of temperature history for the protein's tendency to gel. The total temperature exposure of protein material was calculated from the time of exposure and the temperature. The time-temperature product is called the total temperature exposure (TTE), which was plotted against the viscosity. See FIG. 3.

The results show that the TTE of a protein sample is an important predictor for its tendency to gel. Preferably, the TTE is less than 250° C. hr, more preferably less than 200° C., hr.

TABLE 4
Dynamic viscosity ([Pa · s]) as a function
of TTE for all data in Examples 1-3.

| Heat treatment (deg. C.) | Heat time (h) | TTE | viscosity (Pa*s) |
|---|---|---|---|
| 35 | 4 | 140 | 0.0 |
| 39 | 4 | 156 | 0.2 |
| 43 | 4 | 172 | 18.4 |
| 30 | 3 | 90 | 0.2 |
| 30 | 6 | 180 | 0.5 |
| 30 | 9 | 270 | 15.3 |
| 33 | 3 | 99 | 0.7 |
| 33 | 6 | 198 | 6.1 |
| 33 | 9 | 297 | 42.8 |
| 36 | 3 | 108 | 0.8 |
| 36 | 6 | 216 | 11.6 |
| 36 | 9 | 324 | 62.5 |
| 39 | 3 | 117 | 2.2 |
| 39 | 6 | 234 | 20.6 |
| 39 | 9 | 351 | 83.4 |

### Example 5

Potato was ground and subjected to starch removal with all process steps executed at temperatures between 15 and 25° C. The resulting potato fruit juice was processed using method 1 described in EP 1 920 662 to isolate potato protein patatin as a solution.

The solution was diluted to a concentration of 8 wt. % and adjusted to pH values of 3.5, 3.0, 2.7, 2.3, 2.0 and 1.7 using hydrochloric acid and stored for 6 days at ambient temperature (20-25° C.). The viscosity was determined visually to evaluate gel formation after 5 hours, 22 hours and 5 days.

The results show that pH is a reasonable predictor of gel formation for this protein. In order to remain workable, the pH must be lower than 3.5, preferably lower than 3.0.

TABLE 5
Viscosity at various pH after 5 hours, 22 hours
and 6 days storage at ambient temperature

| pH | 5 hours | 22 hours | 6 days |
|---|---|---|---|
| 3.5 | liquid | gel | gel |
| 3.0 | liquid | gel | gel |
| 2.7 | liquid | liquid | gel |
| 2.3 | liquid | liquid | liquid |
| 2.0 | liquid | liquid | liquid |
| 1.7 | liquid | liquid | liquid |

### Example 6

The potato protease inhibitor obtained in Example 1 was used at a concentration of 16 wt. %. Subsequently, the pH was adjusted to a value of 3.3, and sodium bisulfite was added to reach the indicated concentrations and stored at either ambient temperature or 40° C. The viscosity of the solutions was measured after one day.

TABLE 6
Viscosity at various sulfite concentrations at pH 3.3
after 1 day storage at ambient temperature or 40° C.

| | Ambient temperature | 40° C. |
|---|---|---|
| ppm sulfite | Viscosity Pa*s | Viscosity Pa*s |
| 0 | 0.02 | 0.22 |
| 10 | 0.02 | 0.22 |
| 25 | 0.02 | 0.25 |
| 100 | 0.04 | 1.67 |

The results in table 6 show that viscosity of a potato protein solution is dependent on the temperature. Higher sulfite concentrations result in increased viscosity upon storage, and this effect is stronger at higher temperature.

### Example 7

In a different experiment the effect of sulfite on patatin was investigated, albeit at lower protein concentration, but at higher sulfite levels. The potato protein (patatin) solution obtained in Example 5 was diluted with demineralized water to about 5.3 wt. % concentration. A solution of concentrated citric acid was added under firm stirring ( 1/10 part 40% citric acid). The solution was divided into two parts and a 6% SO₂ solution was added to reach a final sulfite concentration of 0.9 g/L. As a reference, the same volume demi water was added. The pH was set to 1.8-1.9 with 10 M HCl. The final protein content was determined using a CEM Sprint Rapid protein analyzer.

TABLE 7
Viscosity at different sulfite concentrations after 1 day
and 6 days storage at ambient temperatures (20-25° C.)

| | Protein | | | |
|---|---|---|---|---|
| Sample | concentration | Sulfite | 1 day | 6 days |
| 1 | 5.30% | 900 ppm | Liquid | Gel |
| 2 | 5.37% | 0 ppm | Liquid | Liquid |

The results clearly show the negative influence of the presence of sulfite in the stability of a patatin protein solution.

1. A method for isolating native potato protease inhibitor, which method comprises
at least the process steps of
a) processing a potato to obtain potato processing water comprising native potato protease inhibitor;
b) isolating a native potato protease inhibitor from said potato processing water to obtain an aqueous solution of pH 2.5-4.0 comprising at least 5 wt. % of native potato protease inhibitor as the sole type of potato protein,
characterized in that all process steps are performed so as to prevent exposure of native potato protease inhibitor to a temperature higher than 40° C.

2. A method according to claim 1, wherein the method is operated to result in at least at least 5 kg of protein per hour.

3. A method according to claim 1, wherein the pH of the aqueous solution is lower than 3.5.

4. A method according to claim 1, wherein said processing to obtain potato processing water comprises pulping, mashing, rasping, grinding, pressing or cutting of the potato.

5. A method according to claim 1, wherein said processing further comprises a step of starch removal.

6. A method according to claim 1, wherein said isolating of native potato protease inhibitor comprises a step of ultrafiltration, diafiltration, adsorption, precipitation or chromatography.

7. A method according to claim 1, wherein the method further comprises a glycoalkaloid removal step.

8. A method according to claim 1, wherein the aqueous solution is dried to obtain a native potato protease inhibitor powder.

9. A method according to claim 1, wherein the total temperature exposure (TTE) is less than 250° C.·hr.

10. A native potato protease inhibitor obtainable by claim 1, wherein the quantity of disulfide bridges is 4-8 g/kg, expressed as g cysteine present as disulfide bridge per kg protein.

11. A root or tuber native protein protease inhibitor according to claim 10, which has a glycoalkaloid content of at most 200 mg/kg protein.

12. A human food product or an animal feed product comprising a root or tuber native protein according to claim 10.

13. A method according to claim 3, wherein the pH of the aqueous solution is lower than 3.0.

14. A method according to claim 4, wherein the processing comprises a combination with water.

15. A method according to claim 9, wherein the total temperature exposure (TTE) is less than 200° C.·hr.
